(19) United States
(12) Patent Application Publication
(10) Pub. No.: US 2022/0061954 A1
(43) Pub. Date: Mar. 3, 2022

(54) ADVANCED PROTECTIVE CLEAR DOME SHIELD FOR OPTICAL INSTRUMENTS

(71) Applicant: University of Florida Research Foundation, Inc., Gainesville, FL (US)

(72) Inventor: Brian Madow, Gainesville, FL (US)

(21) Appl. No.: 17/402,771

(22) Filed: Aug. 16, 2021

Related U.S. Application Data
(60) Provisional application No. 63/073,143, filed on Sep. 1, 2020.

(51) Int. Cl. A61B 90/20; A61B 3/13

(52) U.S. Cl. CPC A61B 90/20; A61B 2560/06; A61B 3/13

(57) ABSTRACT

Disclosed herein is a dome shield for biomicroscopes and/or other optical instruments. The dome shield is rigid and durable as well as optically clear and prevents respiratory droplets from contacting the face and head of the instrument operator during eye exams. The dome shield has a smooth surface and is removable from the instrument for cleaning and disinfection. In a further aspect, the dome shield allows for the adjustment of the interpupillary distance of the instrument oculars without removal of the dome shield. Also disclosed are adapters or seals for holding the dome shield in place and preventing the transmission of aerosols or droplets at the point of attachment of the dome shield to the optical instrument.

### CROSS REFERENCE TO RELATED APPLICATIONS

This application claims the benefit of U.S. Provisional Application No. 63/073,143 filed on Sep. 1, 2020, which is incorporated herein by reference in its entirety.

### BACKGROUND

SARS-CoV-2 is a highly contagious, single-stranded RNA coronavirus affecting multiple organ systems and processes in the body. Millions of individuals worldwide have been infected, with many requiring hospitalization, supplemental oxygen, anticoagulant drugs, and other supportive therapies, while hundreds of thousands of infected patients, especially the elderly and those with preexisting comorbidities, have ultimately died of COVID-19, the disease caused by SARS-CoV-2. Common early symptoms include fatigue, fever, dry cough, myalgia, diarrhea, and anosmia, with later pulmonary involvement and occasionally sever complications including acute respiratory distress syndrome, arrhythmia, and shock. This highly-transmissible virus is believed to be primarily spread through contact of respiratory droplets and aerosols, including those from presymptomatic and asymptomatic individuals, with mucous membranes.

Although numerous patients have avoided routine eye care visits during the COVID-19 pandemic, optometry offices have remained open so that their patients can be evaluated for new and/or updated prescriptions for corrective vision; undergo check-ups for conditions such as, for example, macular degeneration, cataracts, diabetic retinopathy, glaucoma, and the like; or have injuries and/or acute eye diseases assessed and treated in order to avoid permanent eye damage and/or loss of vision. Many eye care practices have implemented measures such as enhanced cleaning and sanitization, have increased time between appointments to minimize building or office occupancy, and have mandated the wearing of facial masks by employees and patients alike.

However, during a typical eye exam, an ophthalmologist, optometrist, or other technician is normally positioned approximately 13 inches (33 cm) from the face of a patient when using a slit lamp (biomicroscope) or other optical instrument, or only a fraction of the 6 feet suggested by the Centers for Disease Control and Prevention for social distancing to reduce coronavirus transmission. Although eye care offices may screen patients for COVID-19 symptoms prior to appointments, risk to practitioners is still high due to the number of patients seen during any given day, especially given the infectiousness of asymptomatic and presymptomatic individuals.

Surgical masks, though widely used, are largely deemed to be insufficient protection, especially at close distances. N95 masks offer a higher degree of sealing and filtering, but when used alone still do not ensure full protection. Although patient face masks have been advocated for use to prevent the spread of secretions, in many facilities, masks are manufactured in-house from off the shelf barrier materials with uncertain durability and barrier capability. Further, most patients have no experience of wearing masks. They may find the mask uncomfortable, uncover the nose, or completely remove the mask while in the clinic. Additionally, the mask may cause the patient's breath to fog the indirect lens of the biomicroscope or another optical instrument component while being used for a retinal exam. If significant haze occurs, this may decrease the quality of the exam. If an examiner is also wearing a mask, additional fogging of the oculars can occur, yielding frustration and a limited exam.

The face shield has been suggested as an additional protective barrier. However, the material of a typical face shield, although transparent, is not perfectly optically clear. Since the shield covers the upper part of the face including the eyes, using the slit lamp while wearing a face shield can be challenging. These shields, used together with face masks, create a complicated and inferior optical environment due to fogging and material bending, leading to shifting and distortion of the observed images.

A different solution is to use a barrier between the patient and the examiner. Many slit lamps have been fitted with a piece of transparent plastic attached loosely to the console of the slit lamp; these have been termed breath shields and have been used regularly for more than 30 years. These are generally small, flat sections of plastic of inadequate size to protect either patient or optical instrument operator from infectious droplets or aerosols.

With the advent of the SARS-CoV 2 pandemic, some shield designs circulated on the Internet incorporate plastic document folders. However, these typically lack complete transparency, which can hinder usage, especially when attempting to perform procedures at the slit lamp. Using an applanation tonometer in conjunction with a shield constructed from disposable plastic items is also challenging. Since the disposable plastic material is neither rigid nor very smooth, this may impede effective cleaning and disinfection. Furthermore, geometrically, a flat item such as a semi-rigid plastic sheet cannot be simultaneously curved in two different directions; thus, temporary or disposable shields leave at least two opposing sides flat and both practitioner and patient thereby unprotected from aerosol transmission.

What is needed is a rigid, optically transparent shield for biomicroscopes and other optical instruments. Ideally, the shield would be curved on all sides, would be rigid and durable, and could be removed from the instruments for cleaning and/or disinfection prior to reuse. The shield would further allow for adjustment of interpupillary distance for the instrument operator and would include a seal at the point of attachment to the instrument such that aerosols are blocked from passage in both directions. The shield would additionally reduce or eliminate transmission of SARS-CoV 2 and other bacterial and viral diseases during eye exams. These and other needs are addressed by the present disclosure.

### SUMMARY

Disclosed herein is a dome shield for biomicroscopes and/or other optical instruments. The dome shield is rigid and durable as well as optically clear and prevents respiratory droplets from contacting the face and head of the instrument operator during eye exams. The dome shield has a smooth surface and is removable from the instrument for cleaning and disinfection. In a further aspect, the dome shield allows for the adjustment of the interpupillary distance of the instrument oculars without removal of the dome shield. Also disclosed are adapters or seals for holding the dome shield in place and preventing the transmission of aerosols or droplets at the point of attachment of the dome shield to the optical instrument.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals and/or labels designate corresponding parts throughout the several views.

FIG. 1A shows a biomicroscope fitted with a clear dome shield according to one aspect of the present disclosure. FIG. 1B shows the clear dome shield fitted on a PVC base constructed for droplet testing.

FIG. 2 shows a clear dome shield according to one aspect of the present disclosure in use during a patient eye exam.

FIG. 3 shows a closer view of the point of attachment of the dome shield to a biomicroscope including rubber ring (indicated by arrow) for securing the dome shield in place.

FIG. 4A shows an exemplary clear dome useful as a dome shield as disclosed herein. FIG. 4B illustrates the degree of optical clarity that can be expected from the disclosed dome shields.

FIG. 5A provides a cross-sectional view of an exemplary dome shield as described herein showing inner diameter, outer diameter, height, and thickness. FIGS. 5B-5C show a schematic of an exemplary dome shield including a lip around the outer edge of the dome shield.

FIGS. 6A-6D show droplet splash patterns for a small flat shield (FIG. 6A), a medium flat shield (FIG. 6B), a large flat shield (FIG. 6C), and an exemplary dome shield of the present disclosure (FIG. 6D). Images have been enhanced to reveal the finest degree of staining.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a rigid plastic material,” “a disease transmitted by aerosols,” or “an optical instrument,” include, but are not limited to, combinations or hybrids of two or more such rigid plastic materials, diseases transmitted by aerosols, or optical instruments, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

“Slit lamp,” “biomicroscope,” and related terms (e.g., “slit lamp biomicroscope”) are used herein to refer to an instrument for eye exams. Biomicroscopes are low-power microscopes accompanied by a light source having a narrow beam (i.e., “slit lamp”). A biomicroscope can be used to examine several internal structures of the eye, especially when dilated, including the retina and optic nerve. Biomicroscopes also see some use in contact lens exams. A typical slit lamp operator sits about 13 inches (33 cm) from the patient being examined. In one aspect, disclosed herein is a clear dome shield for use with biomicroscopes and other optical instruments.

“Oculars” as used herein refers to the pair of eyepieces used by the operator of a slit lamp or another optical instrument. A typical ocular includes a housing (usually barrel-shaped or cylindrical), which contains one or more lenses or lens elements. The positions of oculars can be adjusted for the “interpupillary distance” of the optical instrument operator, or the distance between the centers of the two eyes, in order to enhance focus and maximize three-dimensional viewing of the structures of the eye.

A “dome” as used herein refers to a hollow, rounded structure having a circular base. In one aspect, a dome is a hemisphere. In a further aspect, the dome shield disclosed herein is a hollow hemisphere having a convex outer or exterior surface and a concave inner or interior surface.

As used herein, “transparent” refers to a material through which light is transmitted without scattering, refraction, or other distortion. Objects observed through a transparent object can be seen clearly. A transparent object can be colorless (i.e., clear) and is not cloudy. “Optical clarity,” meanwhile, refers to a property of materials through which vision is clear and accurate in different lighting conditions and at different angles. In some aspects, “transparency” and “optical clarity” can be used interchangeably.

In one aspect, the dome shields disclosed herein are “rigid.” In a further aspect, “rigid” refers to the stiffness, hardness, and or lack of flexibility and/or deformability. In another aspect, the dome shields disclosed herein are “durable.” In one aspect, “durable” refers to the ability of an object to resist wear, abrasion, and the like.

As used herein, “smooth” refers to a surface free from projections, indentations, or other irregularities of texture. In one aspect, the dome shields disclosed herein are substantially smooth.

Respiratory “aerosols” and “droplets” are created when an individual talks, breathes, coughs, sneezes, or otherwise forcefully exhales (i.e., when air is expelled over a layer of fluid such as, for example, saliva in the mouth or nasal mucus). In one aspect, “aerosols” are small enough and light enough that they may remain suspended in the air for long periods of time due to buoyant forces, while “droplets” tend to be larger and to sink. In individuals who have bacterial or viral infections, aerosols and/or droplets may contain virions or bacteria and are capable of infecting those who come into contact with the aerosols or droplets. Different size ranges for aerosols and droplets have been given by different authorities. In one aspect, a droplet or droplet particle has an average diameter of greater than about 5 μm, while an aerosol particle has an average diameter of less than about 5 μm. In an alternative aspect, a droplet or droplet particle is from about 10 μm to about 100 μm in diameter, while an aerosol particle is less than about 10 μm in diameter.

### Dome Shield With Optical Clarity

In one aspect, disclosed herein is a dome shield for use with optical instruments such as, for example, biomicroscopes. In a further aspect, the dome shield can be configured for use with any brand or model of biomicroscope or other optical instrument. In another aspect, the dome shield completely surrounds the face of the examiner. In still another aspect, the dome shield prevents respiratory droplets and/or aerosols that may contain infectious particles, wherein the droplets and aerosols are generated by a patient, e.g., by coughing, sneezing, talking, or simply breathing, from reaching the examiner's face and/or head.

In another aspect, the dome shield is advantageous over previously known breath shields for biomicroscopes and other optical instruments. In one aspect, a flat breath shield, or one curved in two dimensions only (e.g., top and bottom, or on the sides) does not provide complete protection to the examiner and can allow droplets and aerosols to pass the shield on non-curved sides, potentially contacting the examiner as well as condensing on the examiner's side of the shield and dripping down to contaminate instrument surfaces, the instrument table, and/or the examiner's clothing. In one aspect, no condensation forms on the inside or concave surface (examiner's side) of the dome shield. In another aspect, droplets and/or aerosols that condense on the outside or convex surface (patient's side) of the dome shield flow downward and condense at a single point on the inferior portion of the shield, thereby facilitating cleaning.

In some aspects, the dome shield can optionally be paired with plastic sheeting, a plastic gown, or a similar barrier to prevent droplets and aerosols from reaching the examiner's clothing or hair, the table on which the optical instrument rests, or the examiner's seating, thus reducing the need for cleaning and/or offering additional protection in a high-risk environment.

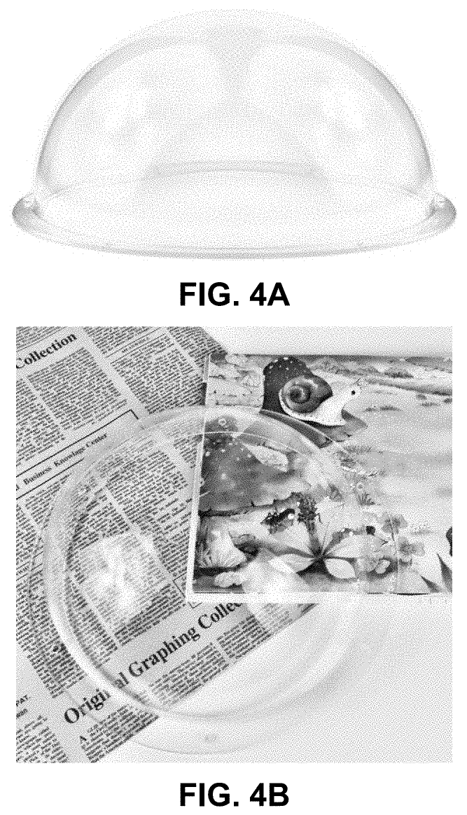

In a further aspect, the dome shield is optically clear. In one aspect, optical clarity of the dome shield is advantageous for reasons including, but not limited to: ability of the examiner to see the patient and provide verbal direction regarding head or body positioning; ability of the examiner to use, without obstruction, an additional device during examination such as, for example, an applanation tonometer; ability to manipulate lenses in front of the patient's eye; ability of patient and examiner to respond to nonverbal communication, cues, gestures, and the like; and combinations thereof. An exemplary dome shield and an illustration of optical clarity of the dome shield are provided in FIGS. 4A-4B.

### Construction and Durability

In one aspect, the dome shield can be made of any optically clear plastic material including, but not limited to, acrylic (e.g., poly(methyl methacrylate) or PMMA), polycarbonate, polystyrene, polyethylene terephthalate, polyethylene terephthalate glycol, or a combination thereof. In one aspect, the dome shield is constructed from a material that will not degrade, deform, or lose rigidity or optical clarity when exposed to water, soaps and detergents, ethanol, isopropyl alcohol, or other common household cleaning and disinfecting solvents.

In another aspect, the dome shield can be made according to any method known in the art including, but not limited to, injection molding, hand forming, pressure and/or vacuum forming, thermoforming, 3D printing, compression molding, or a combination thereof. In one aspect, the dome shield is formed as a dome rather than as a flat sheet and, thus, can remain permanently in the dome shape while not exhibiting any structural stresses or strains (as contrasted with a flat rigid or flexible sheet held into a curved shape under pressure). In any of these aspects, the dome shield is smooth on both the external (convex) and internal (concave) sides. In a further aspect, smoothness and/or lack of texture facilitates cleaning and drying of the dome shield.

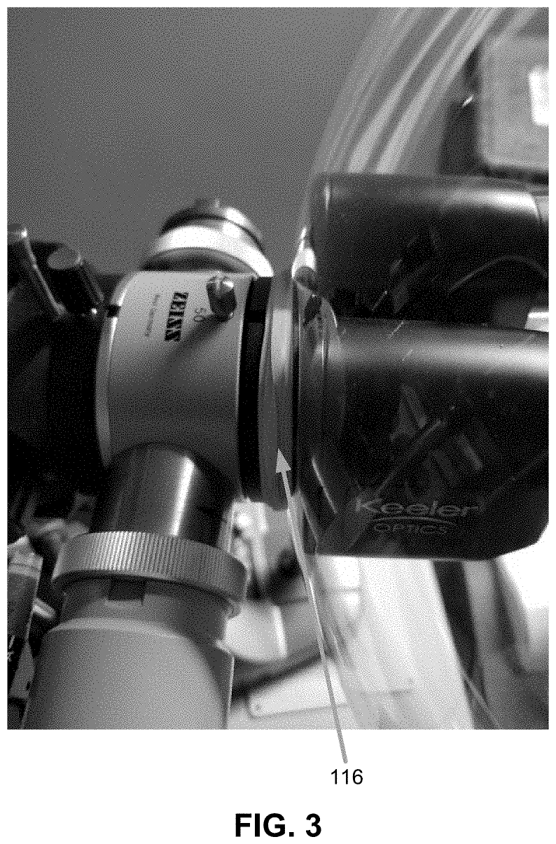

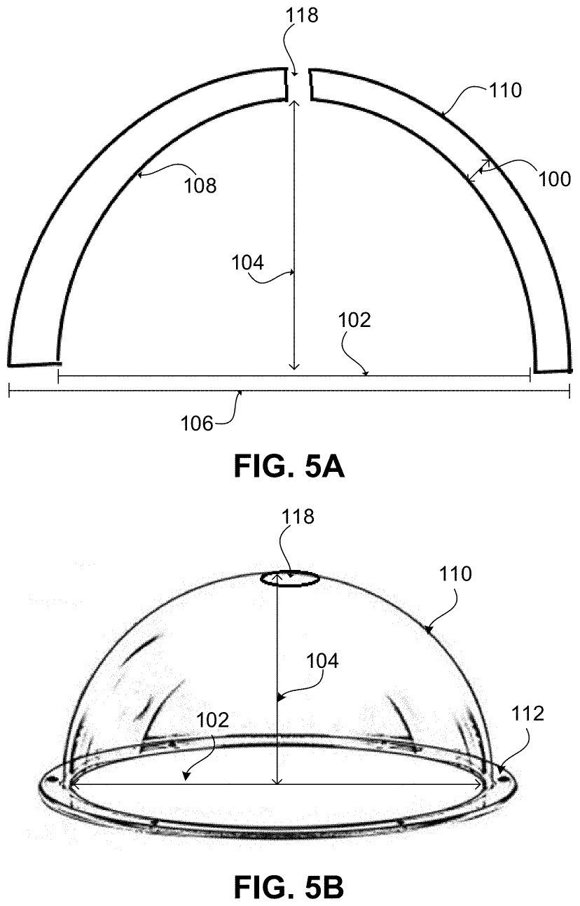

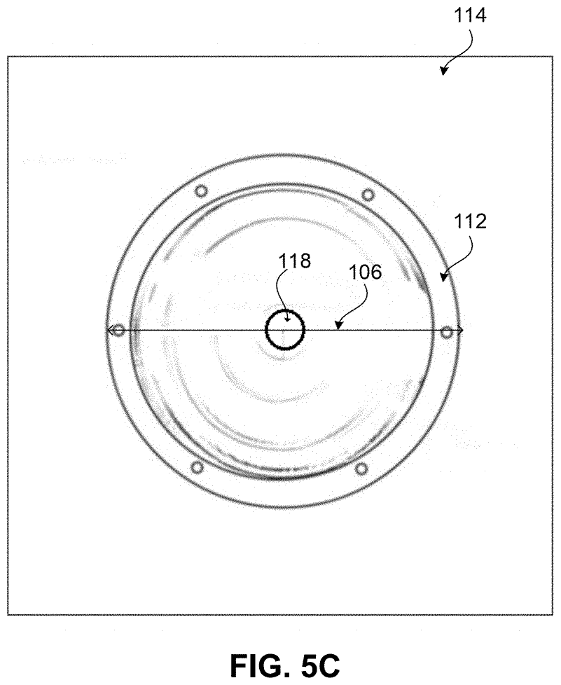

In some aspects, a dome shield can be constructed from an existing clear plastic dome by cutting an aperture 118 (see FIGS. 5A-5C) for receiving the optical instrument (e.g., the lens of a biomicroscope) that fits snugly around the desired portion of the optical instrument. The dimensions and/or shape of the aperture 118 can vary depending upon the dimensions of the optical instrument; however, the dimensions and/or shape of the aperture 118 permit easy removal of the dome shield from the optical instrument. In some aspects, a narrow gap may exist between the shield and the optical instrument. In these aspects, a seal can be used between the shield and the optical instrument and/or on one or both sides of the shield around the point of attachment to the optical instrument. In one aspect, the seal blocks the transmission of aerosols through the gap. In another aspect, the seal can also function to hold the shield in position. In one aspect, a seal at the point of attachment to the optical instrument can be made from rubber, polytetrafluoroethylene (PTFE), silicone, or a combination thereof. An exemplary seal 116 is shown in FIG. 3.

With reference to FIGS. 5A-5C, in one aspect, the dome has a concave surface 108 and a convex surface 110 an inner diameter 102 of from about 20 cm to about 40 cm, or of about 20, 21, 22, 23, 24, 25, 26, 27, 28, 29, 30, 31, 32, 33, 34, 35, 36, 37, 38, 39, or about 40 cm, or a combination of any of the foregoing values, or a range encompassing any of the foregoing values. In one aspect, the dome shield has an inner diameter 102 of about 35 cm to about 36 cm, or about 35.6 cm. In one aspect, the dome has an outer diameter 106 of from about 25 cm to about 45 cm, or of about 25, 26, 27, 28, 29, 30, 31, 32, 33, 34, 35, 36, 37, 38, 39, 40, 41, 42, 43, 44, or about 45 cm, or a combination of any of the foregoing values, or a range encompassing any of the foregoing values. In another aspect, the dome shield has an outer diameter 106 of about 39 cm to about 40 cm, or about 39.6 cm. In one aspect, the outer diameter 106 includes a lip 112 as illustrated in FIGS. 5B-5C having a dimension of from about 0.4 to about 0.8 inches, or from about 10 to about 20 mm, or of about 10, 11, 12, 13, 14, 15, 16, 17, 18, 19, or about 20 mm, or a combination of any of the foregoing values, or a range encompassing any of the foregoing values. In one aspect, the lip 112 is about 20 mm. In any of these aspects, the dome shield has a thickness 100 of about 2 mm to about 6 mm, or about 2, 2.5, 3.0, 3.5, 4.0, 4.5, 5.0, 5.5, or 6.0 mm, or a combination of any of the foregoing values, or a range encompassing any of the foregoing values. In another aspect, dome shield has a thickness 100 of from about 3 mm to about 4 mm, or of about 3 mm, 3.5 mm, or about 4 mm, or a combination of any of the foregoing values, or a range encompassing any of the foregoing values. Without wishing to be bound by theory, a dome shield less than about 3 mm in thickness 100 may be fragile and/or not impact-resistant, while a dome shield thicker than about 4 mm may suffer from reduced clarity. In another aspect, the dome shield has a height 104 as measured from the base of the dome to the apex of the inner surface of the dome shield of from about 10 cm to about 20 cm, or of about 10, 11, 12, 13, 14, 15, 16, 17, 18, 19, or about 20 cm, or a combination of any of the foregoing values, or a range encompassing any of the foregoing values. In one aspect, the dome shield has a height 104 of from about 17 to about 18 cm, or of about 17.8 cm. In one aspect, the dome shield has a surface area of from about 800 cm³ to about 3500 cm³, or of about 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or about 3500 cm³, or a combination of any of the foregoing values, or a range encompassing any of the foregoing values. In one aspect, the dome shield has a surface area of about 1986.3. In some aspects, the dome shield can be surrounded on one or more side with plastic sheeting 114 as shown, for example, in FIG. 5C.

In another aspect, the dome shield is rigid. Rigidity can be advantageous for numerous reasons. In one aspect, a rigid shield cannot be deformed during the process of mounting the shield on the biomicroscope or other optical instrument and/or during the exam when, for example, the examiner must reach around the shield to perform a procedure. In another aspect, a rigid shield easily maintains position when mounted to the optical instrument. In still another aspect, the rigid shield provides a smooth, continuous surface such that any condensation on the patient-facing convex side of the shield collects at a central inferior point.

In one aspect, the dome shield can be removed from the optical instrument for cleaning and/or disinfection. In some aspects, the dome shield can be cleaned while remaining in place on the optical instrument. In a further aspect, the dome shield resists abrasion under conditions of normal use including, but not limited to, removing and replacing the dome shield for cleaning purposes. In another aspect, the dome shield can be scratch-resistant, shatter-resistant, and/or impact-resistant.

In any of these aspects, the dome shield can be cleaned and reused any number of times without requiring replacement. Further in this aspect, the dome shield represents a cost savings over temporary solutions such as, for example, office supplies repurposed as shields, which would require frequent replacement. Also in this aspect, less waste is produced from a single dome shield than multiple temporary shields, and less time is required for cutting the aperture used for mounting a single dome shield versus multiple temporary shields.

### Additional Features and Advantages

In one aspect, an important factor in slit lamp examinations is ensuring the oculars are set at an appropriate interpupillary distance for the examiner. In a further aspect, adjustments may be especially important when multiple examiners are using the same instrument, but routine use of the instrument by the same individual can also slightly misalign the oculars. In a still further aspect, a proper interpupillary distance can maximize 3D viewing of the structures of the eye and enhance focus. In one aspect, a proper interpupillary distance ensures the precise detection of ocular abnormalities at an earlier stage, when, potentially, a wider array of treatment or correction options are available. In one aspect, interpupillary distance of the oculars can be adjusted using the standard instrumental controls without removing the dome shield from the optical instrument.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. An optical instrument comprising a dome shield attached to the optical instrument, wherein the dome shield comprises a concave surface and a convex surface and an aperture for receiving the optical instrument, and wherein the concave surface faces an operator of the optical instrument and the convex surface faces a person being examined by the operator of the optical instrument.

Aspect 2. The dome shield of aspect 1, wherein the dome shield comprises a hollow hemisphere.

Aspect 3. The dome shield of aspect 1 or 2, wherein the dome shield is centered around oculars on the optical instrument.

Aspect 4. The dome shield of any one of aspects 1-3, wherein the concave surface and convex surface are substantially smooth.

Aspect 5. The dome shield of any one of aspects 1-4, wherein the dome shield is optically clear.

Aspect 6. The dome shield of any one of aspects 1-5, wherein the dome shield is configured to be removable from the optical instrument for disinfection.

Aspect 7. The dome shield of any one of aspects 1-6, wherein when respiratory droplets and aerosols are generated facing the convex surface, the dome shield prevents the respiratory droplets and aerosols from reaching the face of the operator of the optical instrument.

Aspect 8. The dome shield of any one of aspects 1-7, further comprising a seal at a point of attachment to the optical instrument, wherein the seal prevents passage of respiratory droplets and aerosols from the convex surface to the concave surface at the point of attachment to the optical instrument.

Aspect 9. The dome shield of aspect 8, wherein the seal comprises rubber, polytetrafluoroethylene, silicone, or a combination thereof.

Aspect 10. The dome shield of any one of aspects 1-9, wherein liquid contacting the convex surface flows to an inferior point of the convex surface of the dome shield.

Aspect 11. The dome shield of any one of aspects 1-10, wherein the dome shield comprises acrylic, polycarbonate, polystyrene, polyethylene terephthalate, polyethylene terephthalate glycol, or a combination thereof.

Aspect 12. The dome shield of any one of aspects 1-11, wherein the dome shield comprises acrylic.

Aspect 13. The dome shield of any one of aspects 1-12, wherein the dome shield is constructed using injection molding, hand forming, pressure forming, vacuum forming, thermoforming, 3D printing, compression molding, or a combination thereof.

Aspect 14. The dome shield of any one of aspects 1-13, wherein the dome shield comprises an inner diameter of from about 22 cm to about 36 cm.

Aspect 15. The dome shield of any one of aspects 1-14, wherein the dome shield comprises an inner diameter of about 35.6 cm.

Aspect 16. The dome shield of any one of aspects 1-15, wherein the dome shield comprises an outer diameter of about 39.6 cm.

Aspect 17. The dome shield of any one of aspects 1-16, wherein the dome shield comprises a thickness of from about 2 mm to about 6 mm.

Aspect 18. The dome shield of any one of aspects 1-17, wherein the dome shield comprises a thickness of from about 3 mm to about 4 mm.

Aspect 19. The dome shield of any one of aspects 1-18, wherein the dome shield comprises a height of from about 10 cm to about 20 cm.

Aspect 20. The dome shield of any one of aspects 1-19, wherein the dome shield comprises a height of about 17.8 cm.

Aspect 21. The dome shield of any one of aspects 1-20, wherein the dome shield comprises a surface area of from about 800 cm³ to about 3500 cm³.

Aspect 22. The dome shield of any one of aspects 1-21, wherein the dome shield comprises a surface area of about 1986.3 cm².

Aspect 23. The dome shield of any one of aspects 1-22, wherein the optical instrument comprises a biomicroscope.

Aspect 24. The dome shield of aspect 23, wherein the dome shield allows for adjustment of interpupillary distance of the biomicroscope without removing the dome shield.

Aspect 25. A protective apparatus for an operator of an optical instrument comprising the dome shield of any of aspects 1-24 and plastic sheeting extending outwards in one or more directions from the dome shield.

Aspect 26. A dome shield for an optical instrument, the dome shield comprising a concave surface and a convex surface and an aperture for receiving the optical instrument.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

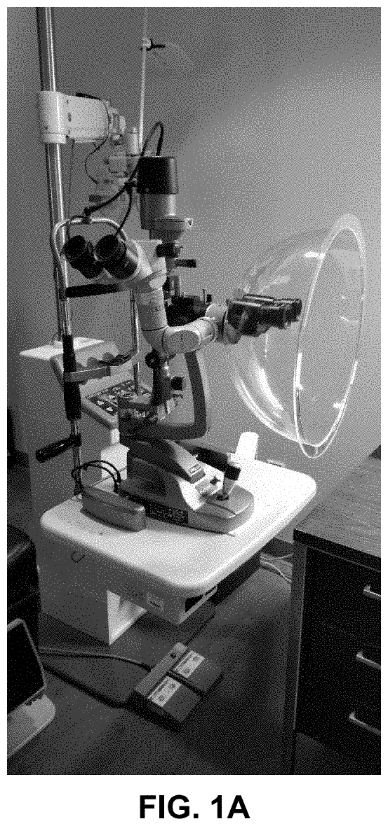

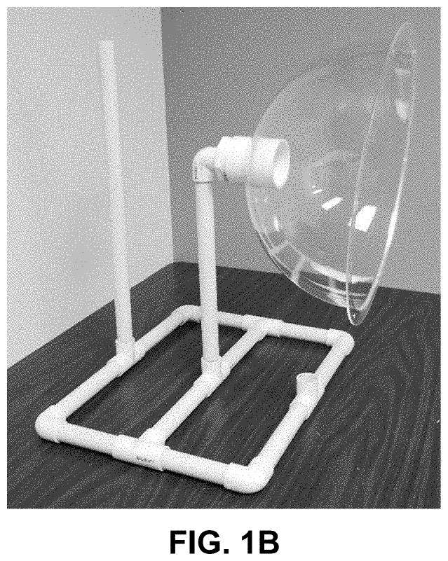

In order to test the level of protection provided to an examiner while using a conventional slit lamp, a trial-stand was constructed (FIG. 1B) to simulate the relative positions of the chin-rest, central biomicroscope or other optical instrument console, and the examiner's face.

Measurements were based on a Zeiss (Oberkochen, Germany) slit lamp (model SL30). The stand was built from commercially available polymerized vinyl chloride (PVC) pipes.

PVC elbow connectors, T-connectors, and 1.9 cm diameter PVC pipes were used to create a base, designed to be wider than the regular slit lamp to provide better stability during the experiment. Vertical pipes were placed at the three positions described above. The central vertical pipe was fitted with an elbow and adapter to accommodate a 5 cm coupling to serve as a holder of the shield being tested. This setting approximates and allows for testing mostly for all slit lamp models, including Zeiss, Keeler, Haag Strait, and others. (FIG. 1B).

### Flat Shields

Three flat shields were tested:
1. A small (standard sized), commercially available breath shield measuring 11.4 cm×20.3 cm (4.5 in width×8 in height)
2. A medium-sized model recreated according to the dimensions of the new “X-large” shield offered by Keeler (Malvern, Pa., USA) measuring 35 cm×35 cm (13.7 in width×13.7 in height)
3. A large-sized model recreated according to instructions distributed by residents and attendings from various institutions and measuring 30.5 cm×45.7 cm (12 in width×18 in height)

The model shields were made from a corrugated white plastic material. A single central opening was cut out towards the superior part of the shield and fitted at the 5 cm coupling as described above.

Black water-based dye was diluted with water in a 1 L spray bottle. The bottle was positioned at the site on the stand corresponding with the chin-rest position on the slit lamp. A white sheet of cardboard was taped vertically to the pipe at a position corresponding to the physician's face.

Each shield was positioned on the stand separately. The liquid was sprayed in the direction of the shield to simulate droplet spread that might occur during a cough or sneeze, and the resulting stain pattern on the test sheet was examined. The test was repeated 3 times in each setting.

### Novel Shield Design

In order to achieve a higher level of protection for the eye-health professional, a more contained environment that would protect more completely from droplets generated by a cough, sneeze, or labored breathing was sought. A dome-shaped shield was designed and 3D modeling was employed to determine the optimal position for the examiner's face and head. The internal radius was selected to allow the examiner to be positioned at the oculars while avoiding contact with any portion of the dome's internal surface, as well as to allow the examiner reach easily around to perform indirect ophthalmoscopy. Optical clarity is essential to allow for a clear view to the patient's eye and to permit the use of the applanation tonometer. Multiple clear plastic spheres with diameters of 9 in (22.9 cm), 11 in (27.9 cm), 12 in (30.5 cm), 13 in (33.0 cm), and 14 in (35.6 cm) were evaluated for potential use in further experiments.

A transparent acrylic plastic hemisphere from a generic manufacturer with an inner diameter of 35.6 cm (14 in), an outer diameter including a lip around the opening of the dome of 39.6 cm (15.6 in), and a thickness of 4 mm was selected. The hemisphere had several factory-made openings at its periphery.

The shield was inserted over the 5 cm holder on the stand and secured in place with 2 rubber bands. The spray bottle was used as described above on the convex part of the shield. White cardboard was positioned inside of the dome at the level of the examiner's face to record any staining.

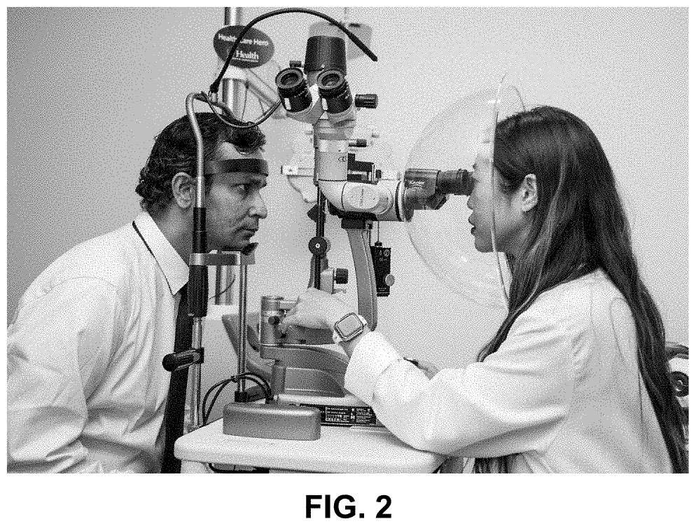

Following testing on the PVC stand as described above, the dome shield was installed on a slit lamp for further evaluation. The dovetail of the Keeler slit lamp ocular block used in these experiments was measured to be 7 cm in diameter. The center of the dome was established using radial treads from each peripheral opening. The center was marked and calipers with a 35 mm radius were used to outline the borders of the opening to be cut. The ocular block was unscrewed from the body of the slit lamp and the dome was inserted through the dovetail with the base facing the examiner. The dome was then secured with two rubber rings (FIG. 3). The oculars were tightened in place holding the dome tightly from the back (FIGS. 1A-1B and 2). The shield was then trialed during eye exams in a retina clinic.

### Example 2: Results

Two patterns were observed in the dye-spraying experiments: larger droplets (>100 μm) and smaller punctate droplets (10-100 μm). The overall stain patterns were evaluated by examining the white target cardboards corresponding to each shield.

### Small Commercial Shield (Standard Size)

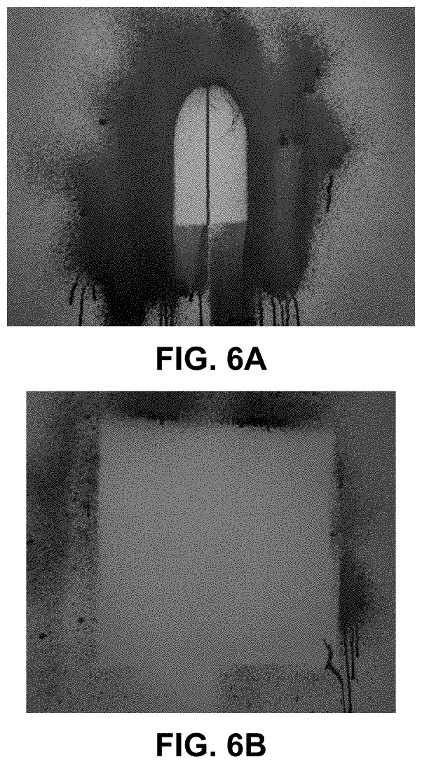

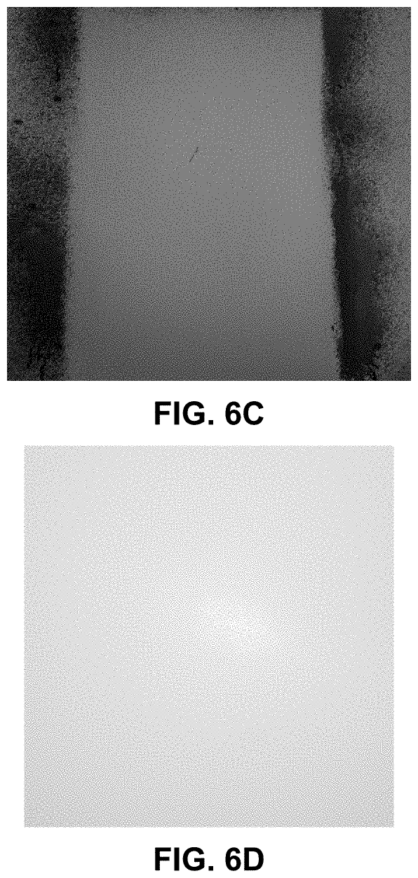

No large droplets stained the testing area centrally. However, large droplets were able to travel beyond the inner border of the shield in all directions. Punctate staining from small droplets was found on the entire surface of the testing area (FIG. 6A).

### Medium Commercial Shield (“X-Large” Shield by Keeler)

No large droplets stained centrally. Staining from small droplets was detected beyond the inner borders of the shield, mostly in the upper half of the target, which corresponds to the position of the examiner's face (FIG. 6B).

### Lame Custom Made Shield

The staining pattern was similar to that of the larger commercially available shield described above. Punctate staining was observed around the edges, with less staining centrally (FIG. 6C).

### Novel Dome-Shaped Shield

No small or large droplets were detected within the area protected by the shield (FIG. 6D). The results were reproducible for each shield.

### Example 3: Conclusions

The tested small, flat shield's surface area was insufficient to protect the face of the examiner. While it may shield the examiner's mouth and the nose directly from large droplets, it neither protects the remainder of the face from large droplets nor shields any part of the examiner's face or upper torso from smaller droplets. The medium and large shields offered an increased area of protection from large droplets, but were also insufficient in protecting against small droplets, including in the area corresponding to the examiner's face. The dome-shaped shield completely protected the testing surface from both small and large droplets.

No shield, except for the dome-shaped shield, fitted tightly around the equipment. Small openings around the oculars allowed for small droplets to travel through the gap, resulting in the observed small droplet staining pattern around the position of the face.

It was noted during the experiment that the sprayed liquid tended to drip along the entire interior width of the shield, which could potentially contaminate the examiner's hands. In contrast, the dome-shaped shield directed all the liquid to a single point inferiorly, which allowed for easier collection and disinfection.

The pattern of sneezing and coughing at the slit lamp is difficult to reproduce. Most patients move away from the chin rest, which changes the path and the direction of droplet spread. However, the simple experiments performed herein confirm and follow the pattern of the droplets shown in other studies.

The dome-shaped shield was sufficiently transparent in clinical testing to allow for facile manipulation of lenses and instruments in front of the eye, as well as unobstructed usage of the applanation tonometer. It does not collect condensation on the examiner's side. The very well-polished surface allows easy cleaning and disinfection. The surface area of the dome was calculated to be 1986.3 cm², which provided more protection than the medium and large flat shields having dimensions of 35 cm×35 cm (1225 cm²) or 30.5 cm×45.7 cm (1394 cm²). It should be noted that a shield width of greater than 35 cm may prevent the examiner from reaching around towards the patient's eye.

The dome shield has only one opening to fit the slit lamp, which is cut very closely to the dimensions of the slit lamp body. This allows for a very tight fit since the adjustment for interpupillary distance is independent of the position of the shield. Additionally, two rubber rings on each side secure the dome and prevent droplets from entering around the mount.

The World Health Organization designates droplet particles as >5 μm and aerosol particles as <5 μm. Other studies have proposed a cutoff of 10 μm for aerosolized particles. Airborne transmission can occur through either droplets (10-100 μm) or through smaller particles that can remain aerosolized (<10 μm). In this experiment, all observed stains, therefore, correspond with droplet-sized particles.

In summary, a new protective clear dome shield was designed. Simulated clinical testing revealed complete protection of the examiner from droplets that may contain viral and bacterial infectious particles. It was found that current flat breath shield designs are ineffective in protecting against droplets spreading from the patient to the physician. The dome-shaped shield established full protection while allowing for ease of use. Additional protection, if desired, may be achieved by fitting the rim of the shield with a plastic gown to provide for face, head, and whole-body protection in very high-risk environments.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. World Health Organization. “WHO Director-General's remarks at the media briefing on 2019-nCoV on 11 Feb. 2020.” 2020 <https://www.who.int/dg/speeches/detail/who-director-general-s-remarks-at-the-media-briefing-on-2019-ncov-on-11-february-2020>(accessed May 1, 2020).
2. Chang D et al. Protecting health-care workers from subclinical coronavirus infection. The Lancet Respiratory Medicine, 2020; 8:e13.
3. Lu C et al, “2019-nCoV transmission through the ocular surface must not be ignored,” The Lancet. 2020, 395:e39.
4. American Academy of Ophthalmology. “Alert: Important coronavirus context for ophthalmologists.” 2020 <https://www.aao.org/headline/alert-important-coronavirus-context> (accessed Mar. 29, 2020).
5. World Health Organization Western Pacific Region, “Practical guidelines for infection control in health care facilities,” 2005, <https://apps.who.int/iris/bitstream/handle/10665/206946/9290222387_eng.pdf?sequence=1&isAllowed=y>(accessed Mar. 29, 2020).
6. Gralton J et al. “The role of particle size in aerosolized pathogen transmission: a review,” J. Infect. 2011, 62:1-13.

What is claimed is:

1. An optical instrument comprising a dome shield attached to the optical instrument, wherein the dome shield comprises a concave surface and a convex surface and an aperture for receiving the optical instrument, and wherein the concave surface faces an operator of the optical instrument and the convex surface faces a person being examined by the operator of the optical instrument.

2. The dome shield of claim 1, wherein the dome shield comprises a hollow hemisphere.

3. The dome shield of claim 1, wherein the dome shield is centered around oculars on the optical instrument.

4. The dome shield of claim 1, wherein the concave surface and convex surface are substantially smooth.

5. The dome shield of claim 1, wherein the dome shield is optically clear.

6. The dome shield of claim 1, wherein the dome shield is configured to be removable from the optical instrument for disinfection.

7. The dome shield of claim 1, wherein when respiratory droplets and aerosols are generated facing the convex surface, the dome shield prevents the respiratory droplets and aerosols from reaching the face of the operator of the optical instrument.

8. The dome shield of claim 1, further comprising a seal at a point of attachment to the optical instrument, wherein the seal prevents passage of respiratory droplets and aerosols from the convex surface to the concave surface at the point of attachment to the optical instrument.

9. The dome shield of claim 8, wherein the seal comprises rubber, polytetrafluoroethylene, silicone, or a combination thereof.

10. The dome shield of claim 1, wherein liquid contacting the convex surface flows to an inferior point of the convex surface of the dome shield.

11. The dome shield of claim 1, wherein the dome shield comprises acrylic, polycarbonate, polystyrene, polyethylene terephthalate, polyethylene terephthalate glycol, or a combination thereof.

12. The dome shield of claim 1, wherein the dome shield is constructed using injection molding, hand forming, pressure forming, vacuum forming, thermoforming, 3D printing, compression molding, or a combination thereof.

13. The dome shield of claim 1, wherein the dome shield comprises an inner diameter of from about 22 cm to about 36 cm.

14. The dome shield of claim 1, wherein the dome shield comprises an outer diameter of about 39.6 cm.

15. The dome shield of claim 1, wherein the dome shield comprises a thickness of from about 2 mm to about 6 mm.

16. The dome shield of claim 1, wherein the dome shield comprises a height of from about 10 cm to about 20 cm.

17. The dome shield of claim 1, wherein the optical instrument comprises a biomicroscope.

18. The dome shield of claim 18, wherein the dome shield allows for adjustment of interpupillary distance of the biomicroscope without removing the dome shield.

19. A protective apparatus for an operator of an optical instrument comprising the dome shield of claim 1 and plastic sheeting extending outwards in one or more directions from the dome shield.

20. A dome shield for an optical instrument, the dome shield comprising a concave surface and a convex surface and an aperture for receiving the optical instrument.
